(19) United States
(12) Patent Application Publication
(10) Pub. No.: US 2004/0260321 A1
(43) Pub. Date: Dec. 23, 2004

(54) Apparatus and method for separating the epithelium layer from the cornea of an eye without corneal pre-applanation

(76) Inventors: Ming-Kok Tai, Lawrenceville, GA (US); James R. Lisk JR., Suwanee, GA (US); Scott M. Hampton, Cumming, GA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/737,533

(22) Filed: Dec. 16, 2003

Related U.S. Application Data
(60) Provisional application No. 60/435,009, filed on Dec. 19, 2002.

(51) Int. Cl. A61F 9/00

(52) U.S. Cl. 606/166

(57) ABSTRACT

An instrument for separating at least a portion of the epithelium from Bowman's layer of a cornea of an eye. The instrument includes a vacuum-operated positioning ring for temporary attachment to the eye so that the cornea is received and exposed in a non-planar configuration and the epithelium is not in contact with any portion of the instrument. In addition, the instrument has a separator with an edge that separates the epithelium of the non-planar cornea from the underlying Bowman's layer as the separator moves along a predetermined path intersecting the cornea. No portion of the instrument contacts the epithelium prior to it being separated from Bowman's layer by the separating edge.

### CROSS-REFERENCE TO RELATED APPLICATION

This application claims the priority benefit of U.S. Provisional Patent Application Ser. No. 60/435,009, filed Dec. 19, 2002, which application is hereby incorporated herein by reference in its entirety.

### FIELD OF THE INVENTION

This invention relates to surgical apparatus and methods and, in particular, to a surgical apparatus and method for separating the epithelium layer of a cornea from the underlying Bowman's layer with minimal trauma to the epithelium layer.

### BACKGROUND OF THE INVENTION

The first microkeratome device for performing corneal resections was developed by Dr. Jose I. Barraquer in 1962. This microkeratome includes a guide ring that is fixed to an eyeball with the aid of a partial vacuum applied through the guide ring. The guide ring immobilizes the eyeball, maintains its tension, and aids in regulating the diameter of the corneal resection. The microkeratome includes a cutting head that is supported within a channel in the guide ring for guided linear movement of the microkeratome across the ring by the surgeon. The cutting head carries a cutting blade that is oscillated transverse the channel as the instrument is moved through the cutting path defined by the channel. In addition, the cutting head carries a removable applanator that compresses the eyeball ahead of the oscillating blade. This permits the blade to cut a lamella having a surface that is lower than and parallel to the compressed planar surface of the cornea. The applanator is interchangeable with similar applanators of differing thicknesses, so as to vary the thickness of the resectioned corneal “disc.”

All known microkeratomes commercially available today applanate, or flatten-out, the cornea of a patient's eye before the cutting blade of the microkeratome creates the flap. This applanation forms a flat surface on the cornea so that the flat cutting blade may create a uniform thickness of cut in the cornea, thereby providing a surgeon with a properly sized flap thickness and diameter. This applanation is a step in the satisfactory operation of all known microkeratomes.

However, applanating the cornea significantly deforms it from its normal curved position to a planar position. Recently, it has been discovered that this applanating deformation can cause epithelial damage or abrasion. When applanating the cornea, it is important to prevent or at least minimize damage to the thin epithelial layer of the cornea. Any damage to the epithelial layer of the cornea can cause discomfort and temporarily diminish the sight of the patient. In this regard, it is believed that if the applanation of the cornea is accomplished in too short a distance of translation, the compression of the cornea can result in damage to the epithelial layer.

Accordingly, recent work has emphasized that the compression of the cornea, and ultimately the full applanation of the cornea, should be accomplished over a sufficient distance of translation in order to minimize the chances for epithelial damage to the cornea. This has been accomplished, for example, by providing a tapered applanator that can be used to gradually compress the cornea. However, a tapered applanator merely reduces damage to the epithelium over flat applanators, and is still not an ideal solution.

Other techniques have shaped the cornea by forcing it into contact with a concave surface with a vacuum prior to initiating the cut. This has been accomplished, for example, by providing a microkeratome having interchangeable inserts with convex, concave, and planar surfaces that engage and compress the cornea for producing a corneal resection of predetermined form and curvature. The inserts are set within a stationary planar member that is fixed to the guide ring. The cutting blade is moved through a cutting path that is parallel to the planar member and defined by a gap between the planar member and the guide ring, and oscillates transverse the path. However, even when used with convex surfaces, the cornea is still subjected to deformation and possible epithelial damage.

Recent developments in laser refractive solutions have resulted in techniques for removing the corneal epithelium without slicing into the stroma. In one such technique, LASEK (Laser Epithelial Keratomileusis), the epithelial layer is separated from the surface of the cornea in a manner that permits the separated epithelial layer to be preserved. Once the exact surface area of treatment is determined, a few drops of a weak alcohol solution are applied to the surface of the cornea and allowed to stay in contact with the epithelium for a few seconds. This weak alcohol solution is then rinsed off the surface of the eye. The function of the weak alcohol solution is to loosen the epithelial layer (which is only about 50 microns thick) so that it can be peeled back in a sheet of epithelial cells, thereby exposing the underlying cornea. However, the use of alcohol causes even more damage to the epithelium than applanation, and is thus not a satisfactory solution to the problem.

More recently, a technique has been demonstrated for separating the corneal epithelium from the underlying Bowman's layer without devitalizing the epithelium with alcohol. A separator such as a plate, wire, or dull blade is used to separate the epithelial layer of an applanated cornea from underlying layers. The separator is much more “blunt” than prior blades and is not sharp enough to “cut” the cornea; rather, it forces a mechanical separation between layers of the cornea. While this technique (known as epiLASIK) typically results in consistent separation of the epithelium from the cornea, it, like known LASIK techniques, has previously required that the cornea be flattened prior to separation of the epithelium, risking abrasion and other trauma to the epithelium.

Therefore, there exists a need in the art for a method and apparatus for separating the epithelium layer of a cornea from the underlying Bowman's layer with minimal trauma to the epithelial layer. It is to the provision of a method and apparatus meeting these and other needs that the present invention is primarily directed.

### SUMMARY OF THE INVENTION

The inventors have determined that, counter to the teachings of conventional surgical methods and microkeratome devices, superior results can be obtained by separating the epithelium of a cornea from the underlying Bowman's layer by moving a separator in contact with a substantially non-applanated cornea. By non-applanated, it is meant that the cornea is in a substantially non-planar configuration.

Accordingly, the present invention provides an instrument for separating at least a portion of the epithelium from the Bowman's layer of a cornea of an eye. The instrument comprises a positioning ring for temporary attachment to the eye. The positioning ring is structured to receive and expose the cornea to be separated in a substantially non-planar configuration and includes a vacuum connection. When a vacuum is applied through the vacuum connection, the epithelium of an eye received in the positioning ring will not be in contact with any portion of the instrument.

The instrument has a separator that separates the epithelium of the cornea from the underlying Bowman's layer as the separator moves along a predetermined path intersecting with the cornea. Preferably, the edge of the separator is not sufficiently sharp to sever Bowman's layer when brought into contact with the eye. In addition, no portion of the instrument will contact the corneal epithelium prior to it being separated from Bowman's layer by the separating edge as the separator moves along the predetermined path.

The present invention also provides a method for separating an epithelium from a cornea of an eye so that an intact Bowman's layer is exposed. The method comprises the steps of fixing a positioning ring to an eye so that the cornea at least partially extends therethrough; moving a separator having a separating edge along a travel path that is generally parallel to the positioning ring and that intersects at least a portion of the cornea so as to separate the epithelium from the cornea while leaving Bowman's layer intact; and retracting the separator to outside the positioning ring. Significantly, the cornea is not flattened prior to moving the separator along the travel path and the cornea is not in a planar configuration when the epithelium is separated from Bowman's layer.

One object of the present invention is to provide an instrument and process for separating the epithelium of a cornea from the underlying Bowman's membrane in such a way that the epithelium can be easily and precisely aligned back-into its original position with minimal trauma following the reshaping of the cornea.

Another object of the present invention is to provide an instrument and process for separating the epithelium of a cornea from the underlying Bowman's membrane in such a way that the epithelium is not contacted by any part of the instrument other than the separating edge of the separator.

A further object of the present invention is to provide an instrument and process that does not obstruct the visual field of the surgeon with an applanator as the separator progresses through the cornea.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 is a cross-sectional view of the first three layers of tissue of the cornea of an eye.

FIG. 2A is a side view of a recently developed applanating separator device having an applanator and a separator slidably engaged with a hand piece and secured to the eye by a vacuum-operated positioning ring.

FIG. 2B is a side view of the applanating separator device of FIG. 2A, showing the applanator beginning to move across and planate the cornea.

FIG. 2C is a side view of the applanating separator device of FIG. 2A, showing the cornea planated by the applanator and the separator moving across and separating the epithelium layer from Bowman's layer.

FIG. 3 is a detail view of the applanating separator device of FIG. 2C, showing the contact between the epithelium and the applanator as the separator moves across the cornea and causes separation of the epithelium layer from Bowman's layer.

FIG. 4 is a side view of a separator assembly according to an exemplary embodiment of the present invention.

FIG. 5 is a side view of a hand piece according to an exemplary embodiment of the present invention, showing the separator assembly of FIG. 4 mounted to the hand piece for use.

FIG. 6A is a side view of the separator assembly of FIG. 4, showing a separator slidably engaged with the hand piece and secured to the eye by a vacuum-operated positioning ring.

FIG. 6B is a side view of the separator assembly of FIG. 6A, showing the separator beginning to move across and separate the unapplanated epithelium layer from Bowman's layer.

FIG. 6C is a side view of the separator assembly of FIG. 6A, showing the separator moving across and separating the unapplanated epithelium layer from Bowman's layer.

FIG. 7 is a detail view of the separator assembly of FIG. 6B, showing the contact between the epithelium and the separator as it moves across the cornea and separates the unapplanated epithelium layer from Bowman's layer.

FIG. 8 is a top view of portions of the separator assembly of FIG. 4 and the hand piece of FIG. 5 after the epithelium has been separated from the eye.

### DETAILED DESCRIPTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

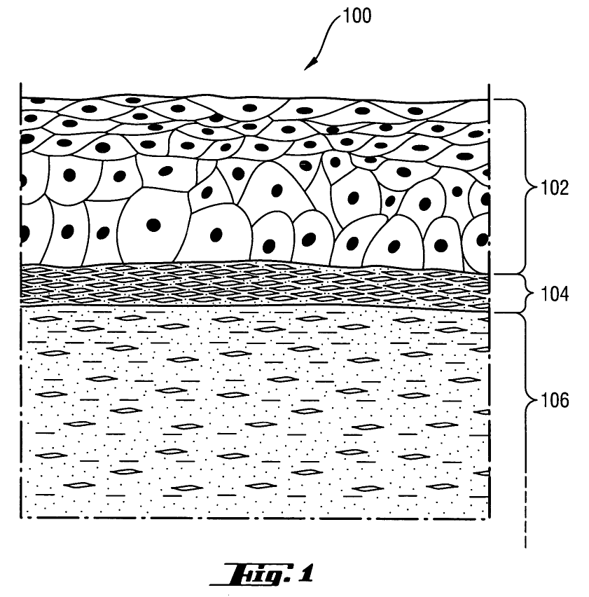

The cornea 100 of the human eye includes five layers, the outer three of which are illustrated in FIG. 1. The outermost layer is known as the epithelium layer 102 and is typically about 50 to 90 microns thick in a healthy adult. The epithelial layer 102 is stratified, typically having 5 to 6 layers of epithelial cells with the layers held together by desmosomes (not shown). Bowman's membrane 104 separates the epithelium from the stroma layer 106. Bowman's membrane 104 is typically about 12 microns thick in a healthy adult, while the stroma 106 is typically about 400 to 450 microns thick and makes up most of the thickness of the cornea. While the exemplary embodiment of the present invention is considered optimal for use upon a human eye, it is understood that such a separator is useful for use on similar animal eyes, including eyes of most mammals and many vertebrates, such as horses, dogs, cats, elephants, sheep, and swine.

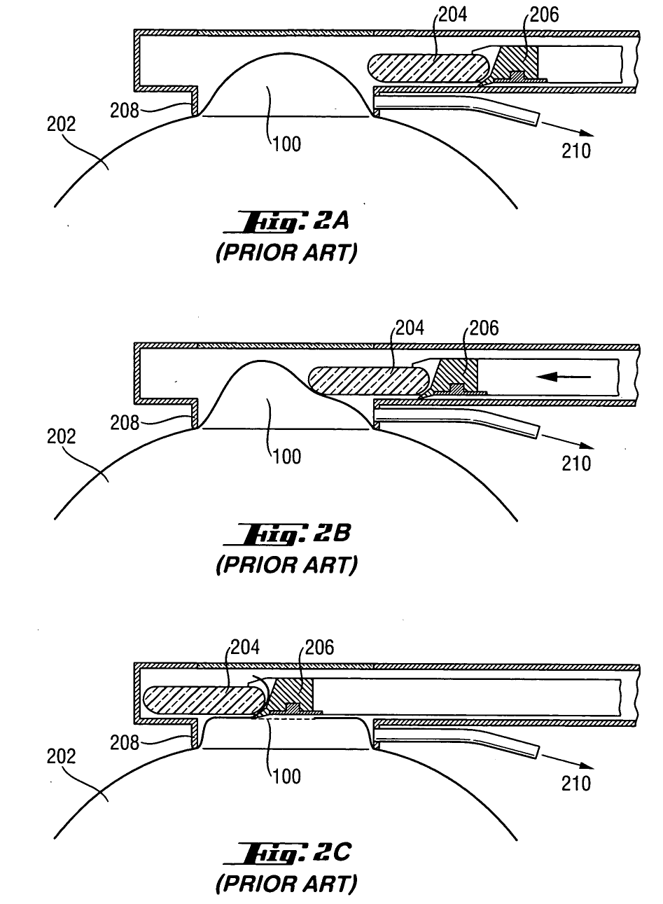

As shown in FIGS. 2A-2C, in a recently developed applanating process and apparatus (used herein to highlight the improvements in the present invention), a separator assembly 200 includes a separator 206 and an applanator 204 in a position forward of the separator. When the eye 202 is placed within a vacuum ring 208 of a hand piece 201 and a vacuum is applied to a vacuum port 210, the surface of the eye 202 is tightened and pulled through the ring 208 to expose the cornea 100 at a position forward of the applanator 204. As shown in FIG. 2A, the separator assembly 200 begins in a first position with the separator 206 located away from the eye 202. Referring now to FIG. 2B, as the applanator 204 moves forward under action of the drive shaft (not shown), the cornea 100 is forced against the undersurface of the applanator 204. This results in a flattening of the cornea 100 before it comes into contact with the separator 206. As the separator assembly 200 moves across the cornea 100 of the eye 202 to a position shown in FIG. 2C, the separator 206 engages the cornea 100 and removes the epithelium layer 102 located at the surface of the cornea 100 of the eye 202.

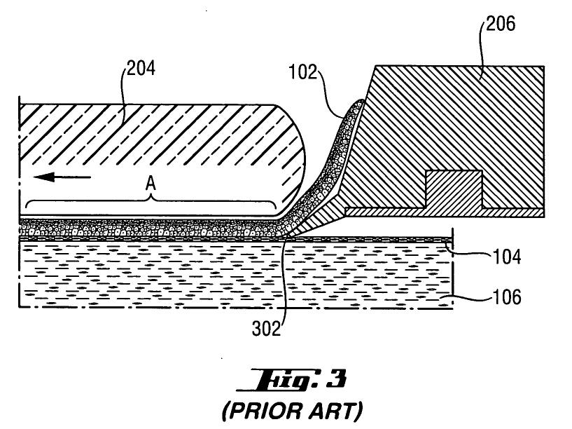

To further demonstrate the operation and disadvantages of applanating separator devices, FIG. 3, is a close-up view of the separation of the epithelium 102 from Bowman's layer 104 in one such process. As the separator 206 and applanator 204 move in the direction of the arrow, the epithelium 102 is under significant pressure and experiences significant friction from the applanator 204 along area “A” until the point at which it is separated from the cornea (point of separation 302). This pressure and friction traumatizes the epithelium. But the applanator 204 is needed in order to flatten the epithelium 102 so that the sharp-edged separator 206 does not cut into Bowman's layer 104.

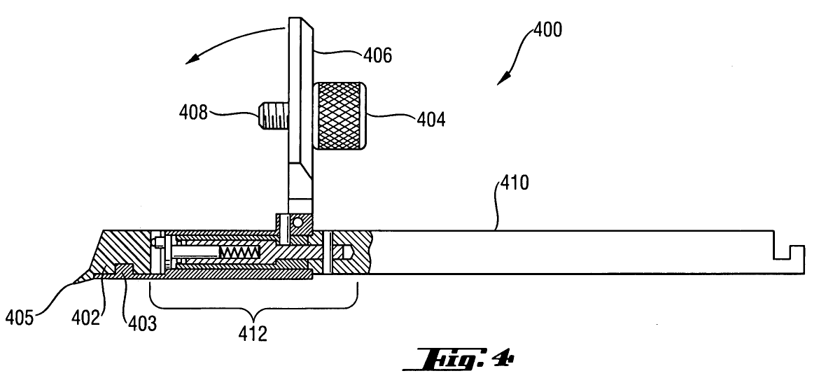

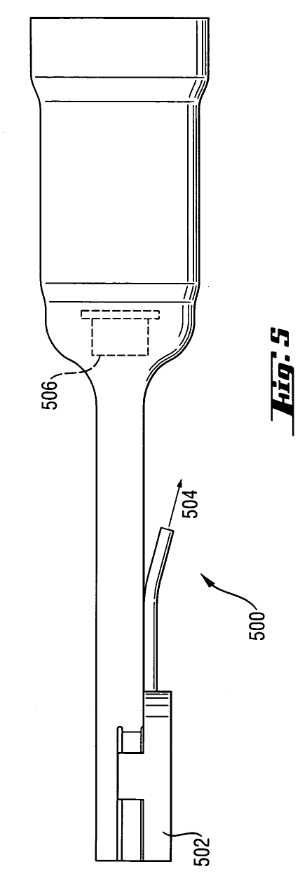

Referring now to FIGS. 4 and 5, a surgical device according to an exemplary embodiment of the present invention comprises a hand piece 500 with a vacuum-operated positioning ring 502 and a separator assembly 400. The separator assembly 400 comprises a separator 402 coupled to a drive shaft 410. The hand piece 500 comprises a motor (not shown) that operably engages the drive shaft 410 and a bushing 506 through which the drive shaft extends. The motor operates to move the separator assembly 400 transversely and to oscillate the separator 402. A vacuum is applied to the vacuum ring 502 through a vacuum port 504 to secure the eye thereto. The vacuum ring 502 is structured such that the epithelium of an eye received therein will not be in contact with any portion of the surgical device. In alternative embodiments, the positioning ring includes other mechanisms for temporary attachment to the eye.

In contrast to typical microkeratome blades, the separator 402 of the exemplary embodiment is preferably not sharp enough to cut into Bowman's layer when used as intended, but rather bluntly separates the corneal epithelium from Bowman's layer as it traverses the cornea. For example, the “blunt” leading edge of the separator 402 of the present invention preferably has a radius of curvature of at least about 5 microns, and no more than about 100 microns. In further preferred embodiments, the radius of curvature of the separator edge is between about 10 microns and about 30 microns, and most preferably is between about 15 microns to about 25 microns. The separating edge 405 of the separator 402 is not so wide that it might reduce the consistency with which the epithelial layer is penetrated. The separating edge 405 preferably is about 5 to 25 micrometers thick, and more preferably about 13 micrometers thick. The separating edge of the separator can be flat, rounded, or even angled, provided, however, that it is not sufficiently sharp to sever Bowman's layer.

The separator 402 can be constructed of any material commonly known in the art, including, stainless steel, ceramics, sapphire, diamond, or plastics, with plastics being preferred. Suitable plastics include, but are not limited to, various grades and formulations of polyetheretherketones (PEEK), poly(methyl methacrylate) (PMMA), acetal homopolymer, polystyrene, methylmethacrylate-acrylonitrile-butadiene-styrene (MABS), and polycarbonate. Preferably, the plastic material of the separator has one or more of the following properties:

a flexural modulus of at least about 1.5 GPa according to ASTM D790-02, more preferably at least about 2.0 GPa, and most preferably at least about 3.0 GPa;

a tensile strength at yield of at least about 25 MPa according to ASTM D638-02, more preferably at least about 40 MPa, and most preferably at least about 50 Mpa;

a Rockwell M hardness greater than or equal to 70 or a Rockwell R hardness greater than or equal to 90, according to ASTM 785-98e1, more preferably a Rockwell M hardness of greater than 90;

a toughness of at least about 1 J/cm², according to ISO 179-1 (15 Dec. 2000) Charpy Impact Test, unnotched at 23° C., more preferably at least about 2 J/cm², most preferably at least about 3 J/cm²; and/or

a Vicat softening point, measured by ASTM D1525-00, of less than 120° C., more preferably at less than 100° C.

Preferably, the motor (not shown) provides two types of motion to the separator assembly 400. The first type of motion is side-to-side oscillation along a path parallel to the separating edge 405 of the separator 402 to assist in the separation process. The second type of motion is longitudinal motion to advance the separator 402 forward across the cornea. The rotational motion of the motor is transferred from the drive shaft 410 to the plunger assembly 412, which translates the rotary motion to oscillations in the separator 402. Thus, by the intermediate action of the plunger assembly 412, the separator 402 is oscillated by the motor. Preferably, the separator 402 can oscillate either transversely, vertically, or longitudinally with frequency ranging from about 10 Hz to about 10 KHz. Electromagnetic or piezoelectric forces on the separator 402 can alternatively provide the oscillation, or external rotating or vibrating wires can provide the oscillation. The separator 402 is preferably oscillated along and supported by a separator support 403 of the separator assembly 400. In alternative embodiments, the motor is provided by a linear actuator, by another actuator that provides one or more of the same or other types of motion, and/or by plurality of motors or other actuators. And in other alternative embodiments, the motion of the motor or other actuator is translated into the motion of the separator by another mechanism such as a gearset, a mechanical linkage, or a different plunger assembly.

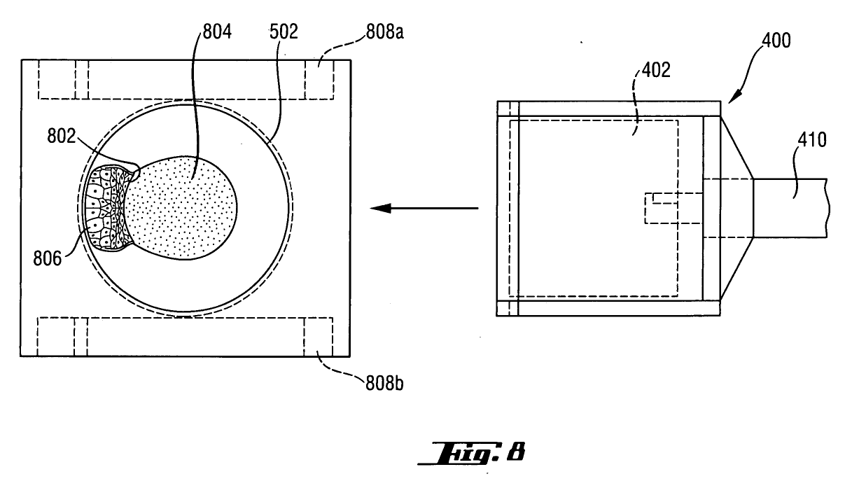

The separator 402 is preferably held firmly within the separator assembly 400 by a separator cover 406, which is preferably hingedly connected to the hand piece 400 moveable in the direction of the arrow in FIG. 4. The cover 406 is secured in place through a locking screw 408, which can be tightened by hand through the locking screw head 404. In addition, the separator assembly 400 is slidably associated with the hand piece 500, for example, by separator guide(s) 808a and 808b such as grooves or tracks within the hand piece 500 (see FIG. 8). The separator guide(s) 808a and 808b are positioned so that the path of separator travel intersects at least a portion of the cornea. In alternative embodiments, the positioning ring has a curvature such that the guides define a path of separator travel that approximates the curvature of the cornea of a typical adult eye, and/or the positioning ring has an adjustment mechanism to permit selectively adjusting the path of separator travel.

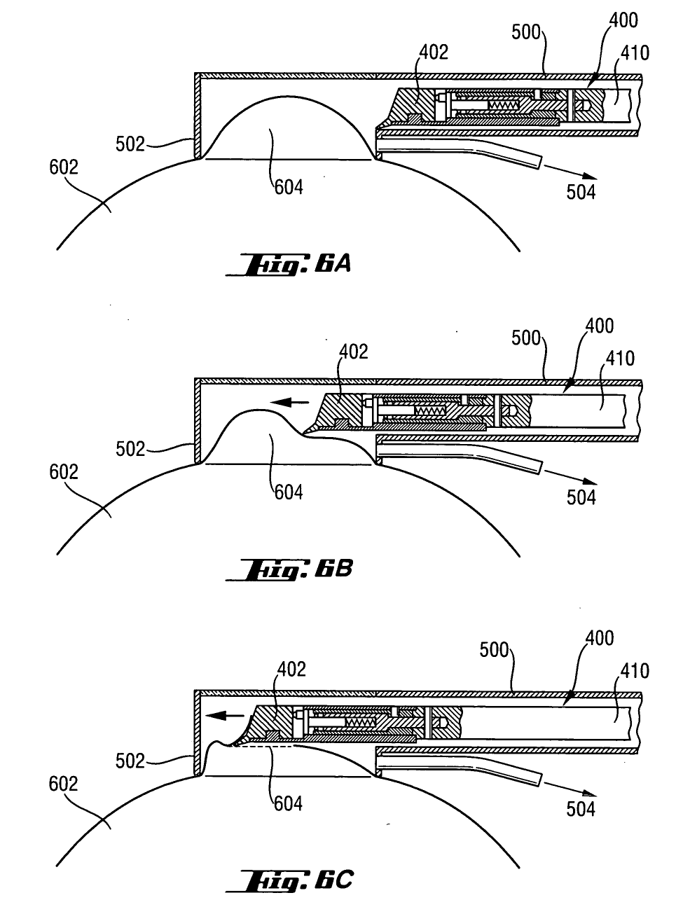

FIGS. 6A-6C show cross sectional side views of an eye 602 of a patient and the surgical device comprising the hand piece 500 associated with the separator assembly 400. (Note that, for simplicity, the separator cover 406 is not shown in FIGS. 6A-6C and the figures are not necessarily drawn to scale.) When the eye 602 is placed within the vacuum-operated positioning ring 502 and a vacuum is applied to the vacuum port 504, the surface of the eye 602 is tightened and pulled through the ring 502 to expose the cornea 604 at a position forward of the separator 402. The corneal epithelium of an eye 602 received in the vacuum ring 502 will not be in contact with any portion of the surgical device.

As shown in FIG. 6A, the separator assembly 400 begins in a first position located away from the eye 602. Since there is no contact—meaning none at all or only minimal and peripheral contact—between the surgical device and the epithelium, the epithelium remains exposed to the ambient atmosphere. Preferably, at least 50% of the epithelium is exposed to the air, and more preferably at least 75%, even more preferably at least 95%, and most preferably greater than 99% of the epithelium is exposed to the air.

Referring now to FIGS. 6B-6C, as the separator assembly 400 moves forward under the action of the drive shaft 410 through the separator guides, the separating edge 405 of the separator 402 is forced against the cornea 604. Because the surgical device has no applanator, there is no flattening of the cornea 604 before it comes into contact with the separator 402. Put another way, no portion of the instrument will contact the epithelium prior to it being separated from Bowman's layer by the separator 402. As the separator assembly 400 moves along the cornea 604 of the eye 602 in the path of separator travel defined by the separator guides, the separator 402 engages the cornea and removes the epithelium layer located at the surface of the cornea of the eye. However, the separator 402 is not sharp enough to excise Bowman's layer during operation of the surgical device.

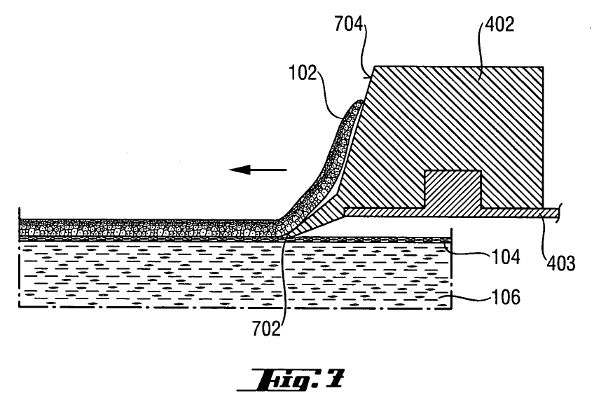

FIG. 7 provides a close-up of the separation of the epithelium 102 from the cornea to expose Bowman's layer 104 according to the present invention. In contrast to the applanating apparatus and method shown in FIG. 3, the present invention avoids significant trauma to the epithelium 102 prior to the point at which the epithelium 102 is separated from the cornea (point of separation 702). In particular, by separating the epithelium 102 without applanation, the pressure and friction cause by prior art applanators is avoided. Instead, as the separator assembly moves in the direction of the arrow, there is no contact with the cornea prior to the engagement of the epithelium 102 by the separator 402 at the point of separation 702. Thus, the only point at which the separator assembly could possibly contact the cornea is at Bowman's layer 104 after the point of separation 702. Bowman's layer 104, however, is much more resistant to abrasions and other trauma than is the epithelium layer 102.

Referring now to FIG. 8, when the separator assembly 400 is retracted from the cornea after separation has occurred, the separated epithelium layer 806 is preferably left partially attached to the cornea of the eye by a hinge 802. The hinge 802 is preferably about 3-4 cm in length, but can differ significantly from this, provided enough of Bowman's layer 804 is exposed to perform laser ablation.

In conventional applanating devices, the separated epithelium 806 typically would have been laid out flat upon the exposed Bowman's layer 804 after the separator assembly was retracted. This is due to the fact that, as shown the applanating device of FIG. 3, the separated epithelium 102 was between the applanator 204 and the separator 206. When the applanator assembly was retracted, the applanator 204 would have exerted a force upon the epithelium 102 in a direction opposite that of the arrow. This would have caused additional trauma to the epithelium 102 and also necessitated using forceps to carefully move the epithelium 102 to the position shown in FIG. 8 (i.e., to where the epithelium already was by using the present invention) prior to laser ablation.

In the present invention however, the separated epithelium 806 will often be left at the hinge 802, away from the exposed Bowman's layer 804. Thus, not only is the additional force upon the epithelium by typical applanators avoided, but also no further manipulation of the epithelium 806 is necessary until after laser ablation is completed and it is replaced back upon the cornea. Even in the event, however, that the epithelium 806 is laid back flat upon the exposed Bowman's layer 804 after the separator assembly is retracted (necessitating its movement prior to laser ablation), there is still a significant advantage in that the epithelium 806 was not, once again, put under pressure or friction by the applanator as it was retracted.

Referring to back to FIG. 7, the separator 402 is used to separate the epithelium 102 of a cornea from the underlying Bowman's layer 104 of an eye of a patient. As the separator 402 is advanced into contact with the eye, the separator edge will cleave the fibrils connecting the epithelium 102 to Bowman's layer 104, but preferably will not slice into Bowman's layer 104. Preferably, the separator 402 pushes the epithelial cells 102 and does not exert a force that could disrupt the intercellular bonds, such as the desmosomes. As the separator edge progresses along the eye, the epithelium 102 is preferably left free to assume an unhindered position and configuration. Often, the epithelium 102 will progress up the front surface 704 of the separator 402 as shown. However, other equally unhindered configurations are possible and desirable.

By not constraining the epithelium 102 during separation, the epithelium encounters minimal stress and strain and will suffer less cell death. This is particularly important when the separator 402 is oscillated. If the epithelium 102 is constrained or otherwise prevented from moving freely (such as being forced against an applanator or other surface post-separation), the oscillatory energy of the separator 402 will be absorbed, at least partially, by the epithelium 102, causing cell disruption or death. However, a freely moving epithelium 102 will not absorb as much energy from the oscillatory movement of the separator 402 and will maintain structural integrity.

Accordingly, the present invention provides a number of advantages over conventional applanating separator devices and techniques. The present invention includes a method and apparatus for separating the epithelium layer of a cornea from the underlying Bowman's layer with no or only minimal trauma to the epithelial layer. In particular, no portion of the apparatus contacts the epithelium prior to it being separated from Bowman's layer by the separating edge, and only the separator contacts it afterward. Significantly, the cornea is not flattened prior to the epithelium being separated from Bowman's layer. And the epithelium can be easily and precisely aligned back into its original position with minimal trauma following the reshaping of the cornea.

While the invention has been described above by reference to various embodiments, it will be understood that many changes and modifications can be made without departing from the scope of the invention. It is therefore intended that the foregoing detailed description be understood as an illustration of the presently preferred embodiments of the invention, and not as a definition of the invention. It is only the following claims, including all equivalents, which are intended to define the scope of this invention.

What is claimed is:

1. An instrument for separating at least a portion of an epithelium from an underlying Bowman's layer of a cornea of an eye, comprising:
(a) a positioning ring structured to receive and expose the cornea when temporarily attached to the eye and structured such that the epithelium received in the positioning ring will not be in contact with any portion of the instrument; and
(b) a separator that separates at least a portion of the corneal epithelium from the Bowman's layer to expose and leave intact the Bowman's layer as the separator moves along a separator guide defining a path of separator travel, the guide being positioned so that the path of separator travel intersects the cornea,
(c) the separator being operatively moveable along the path of separator travel to separate the at least a portion of the corneal epithelium from the Bowman's layer of the cornea, wherein no portion of the instrument contacts the epithelium prior to it being separated from the Bowman's layer by the separator.

2. An instrument as claimed in claim 1, wherein the separator is not sufficiently sharp to sever Bowman's layer when brought into contact with the eye.

3. An instrument as claimed in claim 1, wherein the separator is constructed from a polymeric material selected from the group consisting of polyetheretherketones (PEEK), poly(methyl methacrylate) (PMMA), acetal homopolymer, polystyrene, methylmethacrylate-acrylonitrile-butadiene-styrene (MABS), and polycarbonate.

4. An instrument as claimed in claim 3, wherein the polymeric material has at least one property selected from the group consisting of:
(a) a flexural modulus of at least about 1.5 GPa according to ASTM D790;
(b) a tensile strength at yield of at least about 25 MPa according to ASTM D638;
(c) a Rockwell M hardness of greater than 90 according to ASTM 785;
(d) a toughness of at least about 1 J/cm², according to ISO 179 Charpy Impact Test, unnotched at 23° C.; and
(e) a Vicat softening point, measured by ASTM D1525, of less than 120° C.

5. An instrument as claimed in claim 1, further comprising an actuator operably coupled to the separator for oscillating the separator.

6. An instrument as claimed in claim 1, wherein the positioning ring is vacuum-operated to be temporarily attached to the eye.

7. An instrument as claimed in claim 2, wherein the instrument is free of any structure to applanate the cornea prior to contact with the separator.

8. An instrument for separating at least a portion of an epithelium from an underlying Bowman's layer of a cornea of an eye, comprising:
(a) a vacuum positioning ring for temporary attachment to the eye and structured to receive and expose the cornea in a substantially non-planar configuration; and
(b) a separator that separates at least a portion of the epithelium from the Bowman's layer to expose and leave intact the Bowman's layer as the separator moves along a separator guide defining a path of separator travel, the guide being positioned so that the path of separator travel intersects the cornea,
(c) the separator being operatively moveable along the path of separator travel to separate the at least a portion the epithelium from the Bowman's layer of the cornea, wherein no portion of the instrument contacts or applanates the epithelium prior to it being separated from the Bowman's layer by the separator.

9. An instrument as claimed in claim 1, further comprising an actuator operably coupled to the separator for oscillating the separator.

10. A method for separating at least a portion of an epithelium from a cornea of an eye so that an intact Bowman's layer is exposed, comprising the steps of:
(a) fixing a positioning ring to an eye so that the cornea at least partially extends therethrough;
(b) moving a separator along a travel path defined by the positioning ring and intersecting the cornea to separate the at least a portion of the epithelium from the cornea to expose the Bowman's layer while leaving the Bowman's layer intact; and
(c) retracting the separator from the cornea;
wherein the cornea is not flattened prior to moving the separator along the travel path.

11. A method as claimed in claim 10, wherein the separator is not sufficiently sharp to sever Bowman's layer when brought into contact with the eye.

12. A method as claimed in claim 10, wherein the separator is constructed from a polymeric material selected from the grup consisting of polyetheretherketones (PEEK), poly(methyl methacrylate) (PMMA), acetal homopolymer, polystyrene, methylmethacrylate-acrylonitrile-butadiene-styrene (MABS), and polycarbonate.

13. A method as claimed in claim 12, wherein the polymeric material has at least one property selected from the group consisting of:
(a) a flexural modulus of at least about 1.5 GPa according to ASTM D790;
(b) a tensile strength at yield of at least about 25 MPa according to ASTM D638;
(c) a Rockwell M hardness of greater than 90 according to ASTM 785;
(d) a toughness of at least about 1 J/cm², according to ISO 179 Charpy Impact Test, unnotched at 23° C.; and
(e) a Vicat softening point, measured by ASTM D1525, of less than 120° C.

14. A method as claimed in claim 10, further comprising the step of oscillating the separator as it moves along the travel path.

15. A method as claimed in claim 10, wherein the step of moving the separator includes moving the separator into contact the epithelium before any other movable portion of the instrument.

16. A method as claimed in claim 15, wherein the cornea is curved when it first comes into contact with the separator.

17. A method as claimed in claim 10, wherein the step of retracting the separator includes retracting the separator without physically constraining the separated epithelium.

18. A method as claimed in claim 17, wherein the step of moving the separator includes moving the separated epithelium to one side of the cornea to expose the Bowman's layer, and the step of retracting the separator further includes retracting the separator while leaving the separated epithelium at the one side of the cornea.

19. A method as claimed in claim 10, wherein the step of moving the separator includes moving the separator while leaving at least 50% of the epithelium is exposed to air.

20. A method for separating at least a portion of an epithelium from a cornea of an eye so that an intact Bowman's layer is exposed, comprising the steps of:
(a) fixing a positioning ring to the eye so that the cornea at least partially extends therethrough;
(b) moving a separator having a separating edge along a travel path that is generally parallel to the positioning ring and that intersects the cornea so that the separating edge separates the at least a portion of the epithelium from the cornea to expose the Bowman's layer while leaving the Bowman's layer intact; and
(c) retracting the separator to outside the positioning ring;
wherein the cornea is not in a planar configuration when the epithelium is separated from Bowman's layer.

21. A method as claimed in claim 20, wherein the separator is not sufficiently sharp to sever Bowman's layer when brought into contact with the eye.

22. A method as claimed in claim 20, wherein the separator is constructed from a polymeric material selected from the grup consisting of polyetheretherketones (PEEK), poly(methyl methacrylate) (PMMA), acetal homopolymer, polystyrene, methylmethacrylate-acrylonitrile-butadiene-styrene (MABS), and polycarbonate.

23. A method as claimed in claim 22, wherein the polymeric material has at least one property selected from the group consisting of:
(a) a flexural modulus of at least about 1.5 GPa according to ASTM D790;
(b) a tensile strength at yield of at least about 25 MPa according to ASTM D638;
(c) a Rockwell M hardness of greater than 90 according to ASTM 785;
(d) a toughness of at least about 1 J/cm², according to ISO 179 Charpy Impact Test, unnotched at 23° C.; and
(e) a Vicat softening point, measured by ASTM D1525, of less than 120° C.

24. A method as claimed in claim 20, further comprising the step of oscillating the separator as it moves along the travel path.

25. A method as claimed in claim 20, wherein the step of moving the separator includes moving the separator into contact the epithelium before any other movable portion of the instrument.

26. A method as claimed in claim 25, wherein the cornea is curved when it first comes into contact with the separator.

27. A method as claimed in claim 20, wherein the step of retracting the separator includes retracting the separator without physically constraining the separated epithelium.

28. A method as claimed in claim 27, wherein the step of moving the separator includes moving the separated epithelium to one side of the cornea to expose the Bowman's layer, and the step of retracting the separator further includes retracting the separator while leaving the separated epithelium at the one side of the cornea.

29. A method as claimed in claim 20, wherein the step of moving the separator includes moving the separator while leaving at least 50% of the epithelium is exposed to air.

30. A method of separating corneal epithelium from Bowman's layer, said method comprising moving a blunt separator across at least a portion of the interface between the corneal epithelium and Bowman's layer of the cornea of an eye, while an outer surface of the corneal epithelium remains exposed and free from contact with the separator.

31. A method of cleaving tissue connecting the corneal epithelium and Bowman's layer, said method comprising moving a blunt separator across at least a portion of the interface between the corneal epithelium and Bowman's layer of the cornea of an eye, said method further comprising maintaining intercellular bonds intact within the corneal epithelium and the Bowman's layer.

32. A method of separating corneal epithelium from Bowman's layer, said method comprising separating a flap of corneal epithelium from Bowman's layer, and allowing at least a free end of said flap to remain unconstrained during separation.

33. A disposable separator for separation of corneal epithelium from Bowman's layer, said disposable separator comprising a working edge having a sharpness sufficient to bluntly dissect the corneal epithelium and the Bowman's layer without severing Bowman's layer, while an outer surface of the corneal epithelium remains exposed and free from contact with the separator.

34. The disposable separator of claim 33, wherein said working edge has a radius of curvature of between about 5 microns and about 100 microns.

35. The disposable separator of claim 33, wherein said working edge has a radius of curvature of between about 10 microns and about 30 microns.

36. The disposable separator of claim 33, wherein said working edge has a radius of curvature of between about 15 microns to about 25 microns.
